(19) United States
(12) Patent Application Publication
(10) Pub. No.: US 2012/0196919 A1
(43) Pub. Date: Aug. 2, 2012

(54) EX-VIVO TREATMENT OF IMMUNOLOGICAL DISORDERS WITH PKC-THETA INHIBITORS

(75) Inventors: Maryanne Brown, Mahopac, NY (US); Michael Dustin, New York, NY (US); Alexandra Zanin-Zhorov, Staten Island, NY (US)

(73) Assignees: NEW YORK UNIVERSITY, New York, NY (US); BOEHRINGER INGELHEIM INTERNATIONAL GMBH, Ingelheim am Rhein (DE)

(21) Appl. No.: 13/266,757

(22) PCT Filed: Apr. 28, 2010
(86) PCT No.: PCT/US10/32707

§ 371 Date: Apr. 20, 2012

Related U.S. Application Data
(60) Provisional application No. 61/173,237, filed on Apr. 28, 2009.

(51) Int. Cl. A61K 31/713; A61P 37/06; A61P 17/00; A61K 31/506; A61P 3/10; A61P 1/00; A61K 31/505; A61P 29/00; A61P 19/02

(52) U.S. Cl. 514/44 A; 514/275; 514/274

(57) ABSTRACT

Disclosed is a method for treating a variety of diseases and disorders that are mediated or sustained through the activity of PKC-theta, including immunological disorders and atherosclerosis. Specifically, the invention relates to a method of treating an immunological disorder or atherosclerosis in a patient comprising treating blood from the patient, or a defined component of said blood, with an inhibitor of PKC-theta ex vivo and then re-administering the treated blood to the patient.

### FIELD OF THE INVENTION

This invention relates to a method for treating a variety of diseases and disorders that are mediated or sustained through the activity of PKC-theta, including immunological disorders and atherosclerosis.

### BACKGROUND OF THE INVENTION

The protein kinase C family is a group of serine/threonine kinases that is comprised of twelve related isoenzymes. These kinases are expressed in a wide range of tissues and cell types. Its members are encoded by different genes and are sub-classified according to their requirements for activation. The classical PKC enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKC-theta is a member of the nPKC sub-family. It has a restricted expression pattern, found predominantly in T cells and skeletal muscle. Upon T cell activation, an immunological synapse (IS) composed of supramolecular activation clusters (SMACs) forms at the site of contact between the T cell and antigen presenting cell (APC). PKC-theta is the only PKC isoform found to localize at the SMAC (C. Monks et al., Nature, 1997, 385, 83), placing it in proximity with other signaling enzymes that mediate T cell activation processes. In another study, (G. Baier-Bitterlich et al., Mol. Cell. Biol., 1996, 16, 842) the role of PKC-theta in the activation of AP-1, a transcription factor important in the activation of the IL-2 gene, was confirmed. In unstimulated T cells, constitutively active PKC-theta stimulated AP-1 activity while in cells with dominant negative PKC-theta, AP-1 activity was not induced upon activation by PMA. Other studies showed that PKC-theta, via activation of IκB kinase beta, mediates activation of NF-κB induced by T cell receptor/CD28 co-stimulation (N. Coudronniere et al., Proc. Nat. Acad. Sci. U.S.A., 2000, 97, 3394; X. Lin et al., Moll. Cell. Biol., 2000, 20, 2933). Proliferation of peripheral T cells from PKC-theta knockout mice, in response to T cell receptor (TCR)/CD28 stimulation was greatly diminished compared to T cells from wild type mice. In addition, the amount of IL-2 released from the T cells was also greatly reduced (Z. Sun et al., Nature, 2000, 404, 402). Otherwise, the PKC-theta knockout mice seemed normal and were fertile.

The studies cited above and other studies confirm the critical role of PKC-theta in T cell activation and subsequent release of cytokines such as IL-2 and T cell proliferation (A. Altman et al., Immunology Today, 2000, 21, 567). Thus an inhibitor of PKC-theta would be of therapeutic benefit in treating immunological disorders and other diseases mediated by the inappropriate activation of T cells.

It has been well established that T cells play an important role in regulating the immune response (Powrie and Coffman, Immunology Today, 1993, 14, 270). Indeed, activation of T cells is often the initiating event in immunological disorders. Following activation of the TCR, there is an influx of calcium that is required for T cell activation. Upon activation, T cells produce cytokines, including as IL-2, leading to T cell proliferation, differentiation, and effector function. Clinical studies with inhibitors of IL-2 have shown that interference with T cell activation and proliferation effectively suppresses immune response in vivo (Waldmann, Immunology Today, 1993, 14, 264). Accordingly, agents that inhibit T lymphocyte activation and subsequent cytokine production are therapeutically useful for selectively suppressing the immune response in a patient in need of such immunosuppression and therefore are useful in treating immunological disorders such as autoimmune and inflammatory diseases.

US Patent Application Publication number US 2005/0124640, incorporated herein by reference, discloses compounds of formula (I) that are inhibitors of PKC-theta, useful for treating a variety of diseases mediated by PKC-theta including immunological diseases.

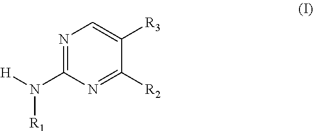

### BRIEF SUMMARY OF THE INVENTION

In a general aspect, the present invention is directed to a method of treating an immunological disorder or atherosclerosis in a patient comprising the treating blood from the patient with an inhibitor of PKC-theta ex vivo and then re-administering the treated blood to the patient.

In another aspect of the invention, the leukocyte fraction from the patient blood is isolated and treated with an inhibitor of PKC-theta ex vivo and then re-administered to the patient.

In another aspect of the invention, Treg cells from the patient blood are isolated and treated with an inhibitor of PKC-theta ex vivo and then re-administered to the patient.

In another aspect of the invention, Treg cells from the patient blood are isolated, induced to grow to generate larger numbers of Treg cells and treated with an inhibitor of PKC-theta ex vivo and then re-administered to the patient.

In another aspect of the invention, the PKC-theta inhibitor is a compound of formula (I)

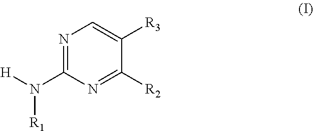

wherein R₁, R₂ and R₃ are as defined herein.

In another aspect, the immunological disorder is selected from inflammatory diseases, autoimmune diseases, organ and bone marrow transplant rejection and other disorders associated with T cell mediated immune response, including acute or chronic inflammation, allergies, contact dermatitis, psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection) and lupus erythematosus.

### BRIEF DESCRIPTION OF THE FIGURES

FIG. 1 a-c

Inhibition of PKC-theta by specific inhibitor, Compound Ia, up-regulates the suppressive function of CD4⁺CD25⁺ T_{reg} cells in vitro.

Treg cells and CD4⁺CD25⁻ Teff (non-Treg) cells were treated with Compound Ia at 0.001-1 microM (a-b) for 30 min, or at 1 microM for 0-60 min (c). Treated cells were mixed with CD4⁺CD25⁻ T (Teff) cells at 1:9 ratio and plated on immobilized anti-CD3 mAb. The supernatants were analyzed for IFN-gamma after 24-48 hours (a and c). Cell proliferation was determined after 96 hours (b). Average of four different experiments is shown.

FIG. 2

Up-regulation of suppressive function by inhibitors of PKC-theta correlates with their IC₅₀'s

Treg were treated with 1 microM PKC-theta inhibitors with different IC₅₀ values as indicated on graph. Treated cells were mixed with CD4⁺CD25⁻ T (Teff) cells at 1:9 ratio and plated on immobilized anti-CD3 mAb. The supernatants were analyzed for IFN-gamma after 24-48 hours. An average of four different experiments is shown. As shown in the graph, the enhancement of suppressive effect on IFN-gamma secretion generally correlates with the potency of the inhibitors.

FIG. 3

Treg were transfected with silent RNA targeting PKC-theta, or with control silent RNA and plated on anti-CD3 mAb. After 48 hours the PKC-theta expression was measured by Western blot analysis.

FIG. 4

Inhibition of PKC-theta by siRNA up-regulates the suppressive function of CD4⁺CD25⁺ T_{reg} cells in vitro.

Treated Treg and non-Treg, or siRNA-transfected Treg were mixed with CD4⁺CD25⁻ T (Teff) cells at 1:9 ratio and plated on immobilized anti-CD3 mAb. The supernatants were analyzed for IFN-gamma after 24-48 hours. An average of four different experiments is shown.

FIGS. 5a-5d

Treatment with PKC-theta inhibitor up-regulates Treg function in vivo.

Colitis was induced in C57BL/10.PL TCR alpha^{-/-}beta^{-/-} mice as described in Methods summary. 5a—numbers of mice were 5 (PBS), 8 (Teff), 7 (Teff/Treg control), 7 (Teff/Treg PKC-theta inhibitor). 5b-Histology slides of distal colon for the different groups. Normal histology is observed in PKC-theta treated mice. 5c, 5d-Freshly purified Treg from healthy donors and RA patients were treated or not with PKC-theta inhibitor for 30 min at 1 microM, washed three times, mixed with CD4⁺CD25⁻ T cells at ratio 1:3, and plated on anti-CD3 mAb. The supernatants were analyzed for IFN-gamma after 24-48 hours. Combined data of three independent experiments are presented. % Treg-mediated inhibition was calculated as: 1−level of IFN-gamma in presence of Treg/level of IFN-gamma in absence of Treg)φ100%. P values were calculated by t-test.

### DETAILED DESCRIPTION OF THE INVENTION

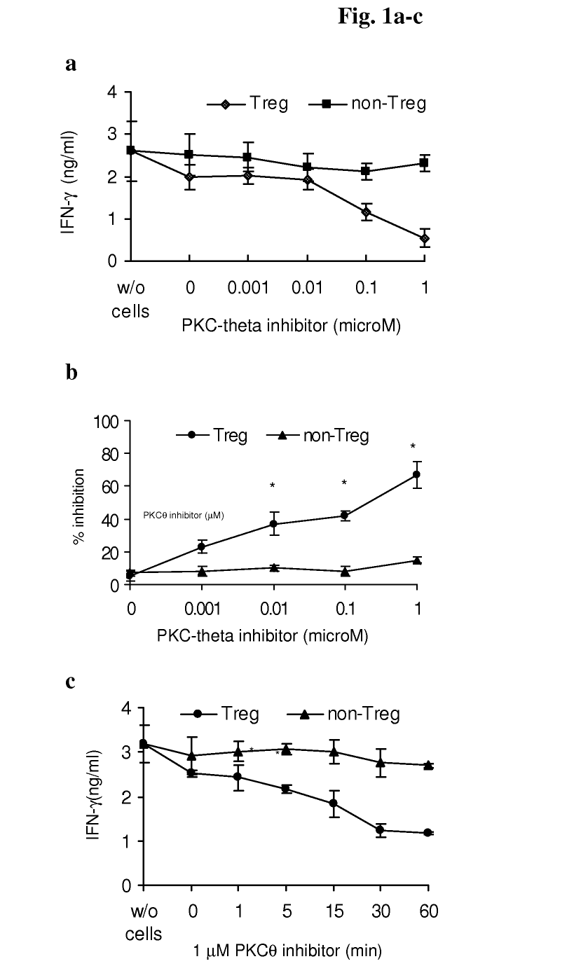

CD4⁺CD25⁺ regulatory T cells (Tregs) suppress the function of CD4⁺ and CD8⁺ effector cells (Teff) through an antigen receptor and cell contact mechanism. Recent studies demonstrated that TCR activation is required for the ability of the Treg cells to inhibit proliferation of responder CD4⁺CD25⁻ T cells in vitro (A. M. Thornton et al., Eur. J. Immunol, 2004, 34, 366). We investigated whether inhibition of PKC-theta may affect the suppressive function of human CD4⁺CD25⁺ Treg cells in vitro. For this purpose, we treated CD4⁺CD25⁺ Treg or CD4⁺CD25⁻ Teff cells (Treg or non-Treg respectively in FIGS. 1a and 1b) with a specific PKC-theta inhibitor, namely, Compound Ia, washed and cultured the cells with untreated Teff cells in a ratio of 1:9 on stimulatory anti-CD3 antibodies. IFN-gamma secretion and cell proliferation were measured after 24 and 96 hours respectively. We found that the PKC-theta inhibitor significantly up-regulated the suppressive ability of Treg cells, but does not induce suppressive activity in Teff that are added with untreated responder Teff (FIGS. 1a and 1b). FIG. 1a shows the inhibitory effect on IFN-gamma production and FIG. 1b shows the effect on cell proliferation. Moreover, the effect of PKC-theta inhibitor on Treg function was time-dependent; maximal levels of enhanced suppressive function were achieved after 30 min of Treg treatment with the compound (FIG. 1c).

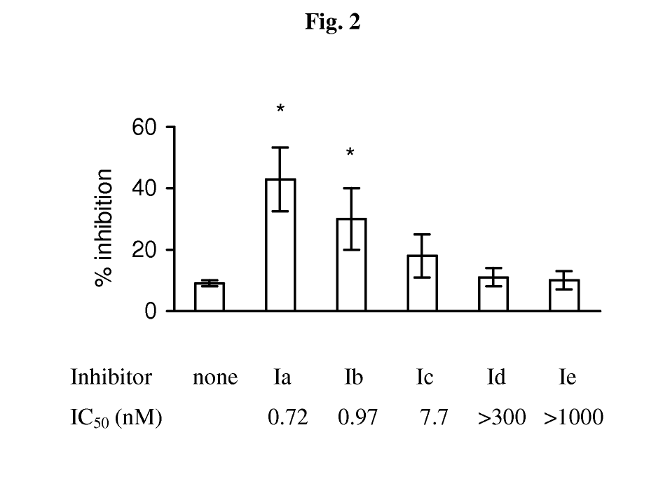

Treatment of Treg cells with analogs of PKC-theta inhibitors with different IC₅₀ values demonstrated the correlation of the suppressive effect with potency of the inhibitor. The PKC-theta inhibitors with IC₅₀≦1 nM significantly up-regulated their suppressive function (FIG. 2) while the effect of the inhibitors with IC₅₀ of 8 nM or greater was not significant. Thus, the ability of PKC-theta inhibitors to boost Treg function is generally correlated with their inhibitory capacity.

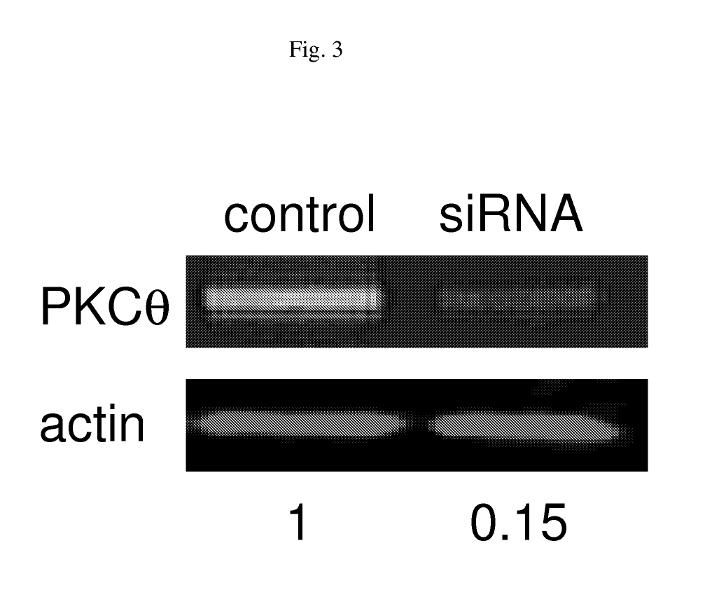

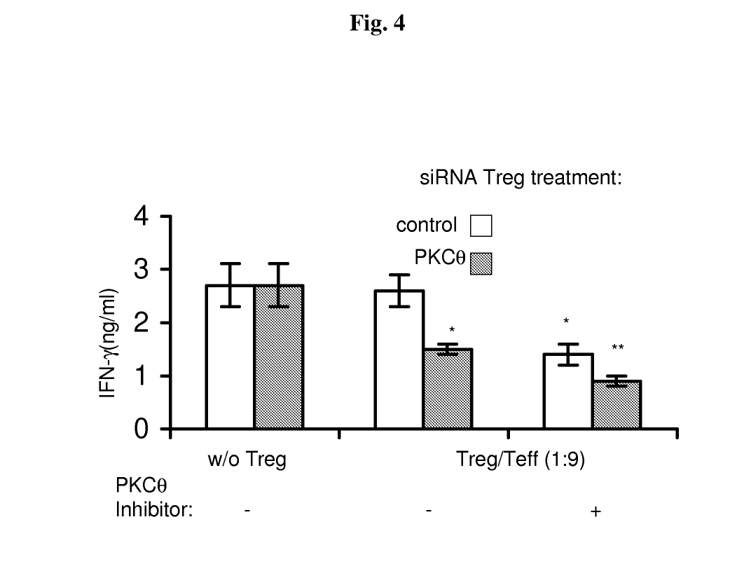

To confirm the conclusion that inhibition of PKC-theta up-regulates the suppressive activity of human Treg, we specifically silenced PKC-theta gene expression using RNA interference (K. K. Srivastava et al., J. Biol. Chem., 2004, 279, 29911). This treatment abrogated the PKC-theta expression in Treg cells by 80% (FIG. 3). Moreover, the ability of Treg cells to inhibit IFN-gamma secretion in Teff cells was increased by specifically silencing the PKC-theta gene, as well as by treatment of Treg cells with PKC-theta inhibitor Ia (FIG. 4). Silencing of the PKC-theta gene in Teff cells resulted in the significant down-regulation of IFN-gamma secretion. In summary, we concluded that inhibition of PKC-theta either by specific inhibitors or by siRNA up-regulates the suppressive function of human Treg cells in vitro.

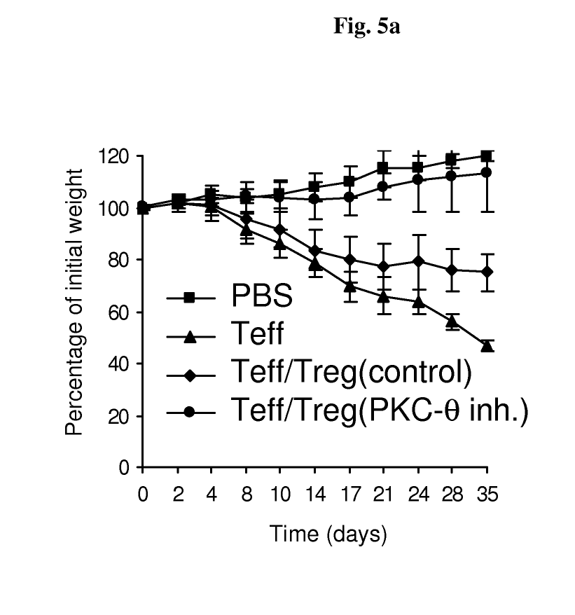

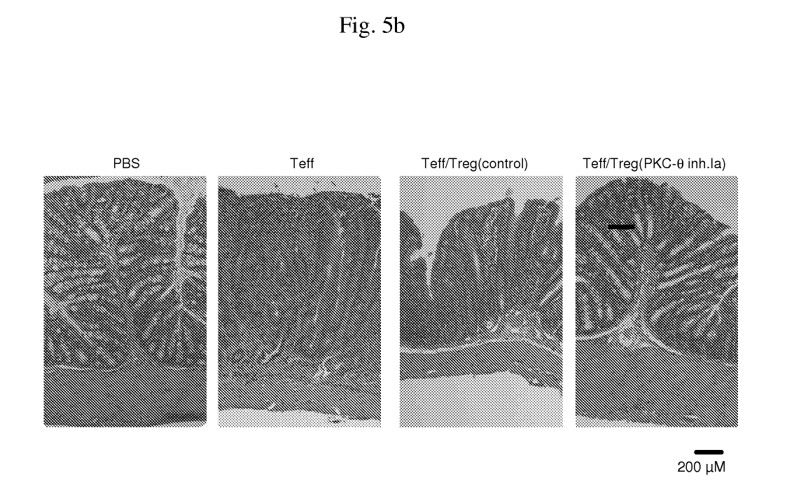

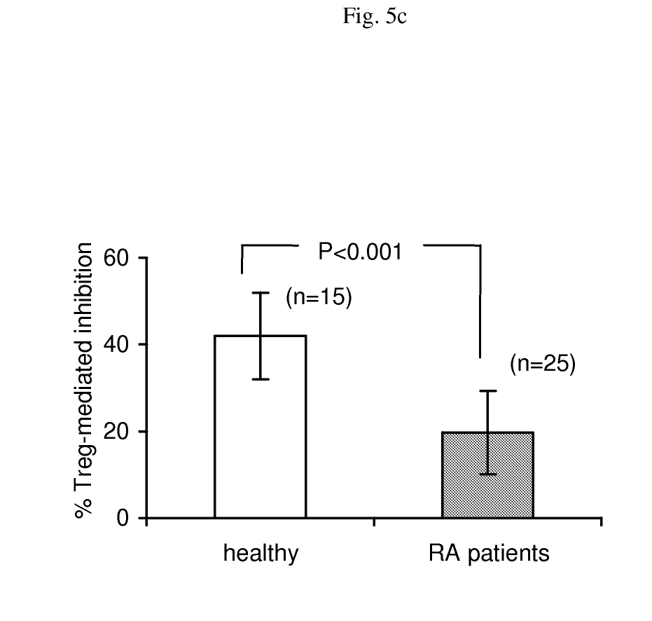

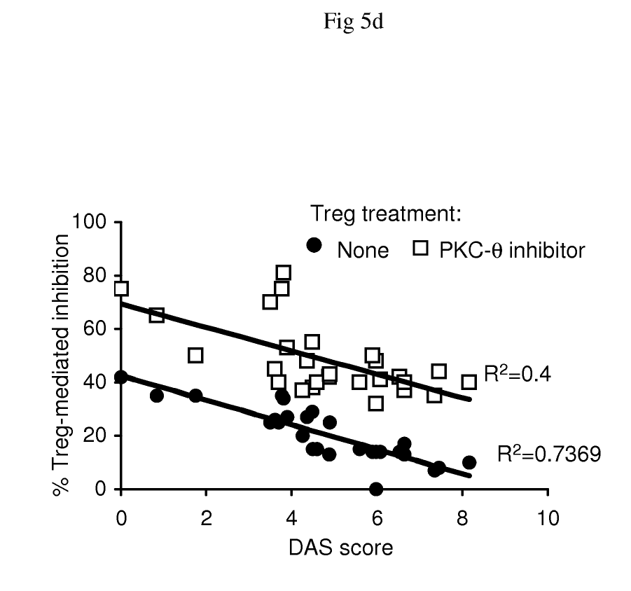

Next, we determined the ability of PKC-theta inhibitor Ia to up-regulate Treg function in vivo using a colitis model in TCR alpha^{-/-}beta^{-/-} mice induced by transfer of effector CD4⁺CD25⁻CD45RB⁺ Teff cells (F. Powrie, et al., J. Exp. Med. 1996, 183, 2669). At a 1:4 ratio of Treg/Teff, PKC-theta inhibitor treated-CD4⁺CD25⁺ Treg cells provided nearly 100% protection of the recipient mice from colitis, as demonstrated by normal weigh gain and normal histology of the distal colon in 7 of 8 mice (FIGS. 5a and 5b). This protection was far superior to that afforded by Treg either left untreated or treated with a much weaker PKC-theta inhibitor at the same Treg/Teff ratio. Thus, inhibition of PKC-theta in mouse Treg significantly up-regulates their suppressive function in vivo.

Rheumatoid arthritis (RA) is a chronic autoimmune disorder that ultimately leads to the destruction of joint architecture. Recent studies in RA patients have suggested that the function of CD4⁺CD25⁺ Treg cells is impaired (Ehrenstein, M. R., et al., J. Exp. Med., 2004, 200, 277). By using CD4⁺CD25⁺ Treg cells, purified from peripheral blood of 25 RA patients with different severities of disease we found that although Treg numbers were comparable with healthy donors, Treg cells demonstrated significantly reduced ability to suppress the production of IFN-gamma from autologous Teff cells compared to healthy donors (FIG. 5c). Moreover, the defective Treg function in RA patients was inversely correlated with the disease active score (DAS score; FIG. 5d). Treg cells from patients with more progressive and active disease (DAS>5) demonstrated about 2-4-fold reduction in Treg-mediated suppression of IFN-gamma from Teff cells, whereas Treg cells from RA patients with moderate or inactive disease (DAS<5) were more effective and suppressed IFN-gamma secretion at levels similar to Treg cells from healthy donors (25-40% inhibition at a Treg/Teff of 1:3). Furthermore, treatment with PKC-theta inhibitor Ia significantly increased the suppressive function of Treg cells purified from all 25 RA patients (FIG. 5d) to levels that comparable with healthy donor-derived Treg cells. These results indicate that inhibition of PKC-theta reverses the defective suppressive function of Treg cells isolated from RA patients.

The application of Treg based adoptive immunotherapy for the treatment of autoimmune diseases has recently become feasible due to improved methods to grow large numbers of Treg in vitro (see review by C. H. June and B. R. Blazar Seminars in immunology, 2006, 18, 78 and also Hippen, et al., Blood 2008, 112: 2847). Possible applications include treatment of Graft-versus-host disease, organ rejection and autoimmune diseases, including multiple sclerosis, systemic lupus erythematosus, ulcerative colitis, Crohn's disease, rheumatoid arthritis and Type 1 diabetes. Isolation and ex vivo expansion of Treg populations for use in adoptive immunotherapy is documented in the literature and known in the art (see also US 2009/0010950 A1, incorporated herein by reference), In the studies above, we have shown for the first time that the inhibition of PKC-theta has potential in Treg based adoptive immunotherapy.

Treg cells have also been reported to have an inhibitory effect on atherosclerosis (P. Aukrust et al., Curr. Atherosclerosis Reports, 2008, 10, 236) and have shown to have an inhibitory effect in a mouse model of atherosclerosis (H. Ait-Oufella et al., Nature Medicine, 2006, 12, 178). Thus, inhibition of PKC-theta in Treg cells which has been shown to boost the suppressive effects of this cell population, should have a beneficial effect in atherosclerosis.

In one embodiment, blood is isolated from a patient having an immunological disorder, the blood is treated ex vivo with an inhibitor of PKC-theta and then infused back into the patient.

In another embodiment, blood is isolated from a patient having atherosclerosis, the blood is treated ex vivo with an inhibitor of PKC-theta and then infused back into the patient.

In another embodiment, the leukocyte fraction of the blood is isolated from a patient having an immunological disorder, the leukocyte fraction is treated ex vivo with an inhibitor of PKC-theta and then infused back into the patient.

In another embodiment, blood is isolated from a patient having an immunological disorder, the Treg cells are isolated and expanded ex vivo, treated with an inhibitor of PKC-theta and then infused back into the patient.

In another embodiment, blood is isolated from a patient having atherosclerosis, the Treg cells are isolated and expanded ex vivo, treated with an inhibitor of PKC-theta and then infused back into the patient.

In another embodiment, peripheral blood mononucular cells and T-cells are separated by plasmapheresis from blood isolated from a patient having an immunological disorder and are treated with an inhibitor of PKC-theta and then infused back into the patient.

In another embodiment, peripheral blood mononucular cells and T-cells are separated by plasmapheresis from blood isolated from a patient having atherosclerosis and are treated with an inhibitor of PKC-theta and then infused back into the patient.

In another embodiment, the PKC-theta inhibitor is a compound of formula (I)

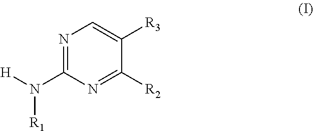

R₁ is aryl-C₁₋₄alkyl or heteroaryl-C₁₋₄alkyl, wherein in each of the C₁₋₄alkyl groups a methylene group may optionally be replaced by —NHC(O)— or —C(O)NH—, and wherein each of the C₁₋₄alkyl groups is optionally substituted by an oxo group or one or more C₁₋₃alkyl groups wherein two alkyl substituents on the same carbon atom of a C₁₋₄alkyl group may optionally be combined to form a C₂₋₅ alkylene bridge, and wherein the aryl group is optionally substituted on adjacent carbon atoms by a C₃₋₆alkylene bridge group wherein a methylene group is optionally replaced by an oxygen, sulfur or —N(R₆)—;
or R₁ has the following structure:

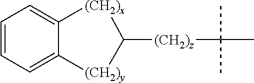

wherein x and y are independently 0, 1, 2 or 3, provided that x+y is 2 to 3, and z is 0 or 1;
wherein “heteroaryl” is defined as pyridyl, furyl, thienyl, pyrrolyl, imidazolyl, or indolyl;
wherein each R₁ group is optionally substituted by one or more of the following groups: C₁₋₆alkyl, Cl, Br, F, nitro, hydroxy, CF₃, —OCF₃, —OCF₂H, —SCF₃, C₁₋₄alkyloxy, C₁₋₄alkylthio, phenyl, benzyl, phenyloxy, phenylthio, aminosulfonyl, or amino optionally substituted by one or two C₁₋₃alkyl groups;
R₂ is selected from the following groups:

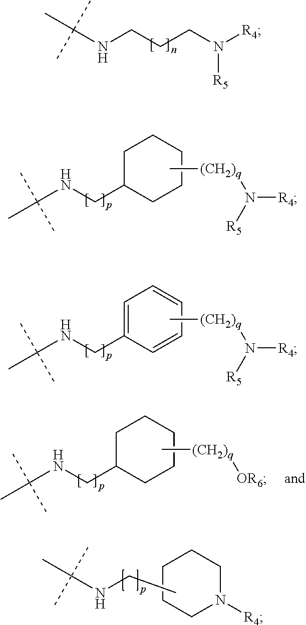

wherein:
n is an integer from 5 to 7;
p is an integer from 1 to 2;
q is an integer from 1 to 2;
R₄ and R₅ are each independently selected from hydrogen, C₁₋₆alkyl, arylC₁₋₆alkyl, or amidino;
R₆ is hydrogen;
R₃ is Br, Cl, F, cyano or nitro;
or a tautomer, pharmaceutically acceptable salt or solvate thereof.

In another embodiment, the PKC-theta inhibitor is any inhibitor of PKC-theta which is disclosed in US Patent Application Publication number US 2005/0124640, all generic and specific embodiments of which are herein incorporated by reference.

In another embodiment, the PKC-theta inhibition is achieved by siRNA or shRNA mediated suppression of PKC-theta and either (a) the siRNA or shRNA is targeted to cells that include Tregs ex vivo followed by infusion of the treated cells into patients or (b) the siRNA or shRNA is directly administered to the patient. In a specific embodiment blood is isolated from a patient, the Treg cells are isolated and expanded ex vivo, treated with an siRNA or shRNA and then infused back into the patient.

Thus, in a specific embodiment the PKC-theta inhibition is achieved by siRNA or shRNA mediated suppression of PKC-theta and the method comprises treating blood from the patient with siRNA or shRNA ex vivo and then re-administering the treated blood to the patient. In a more specific embodiment, the Treg cells from the patient blood are isolated and treated with siRNA or shRNA ex vivo and then re-administered to the patient.

In another embodiment the immunological disorder is selected from psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection), systemic lupus erythematosus

### Experimentals

CD4⁺CD25⁺ Treg and CD4⁺CD25⁻ Teff cells were purified from the peripheral blood of healthy human donors or from 25 patients with Rheumatoid arthritis in different stages (accordingly to disease activity score (DAS)) as described in the literature M. L. Prevoo, et al., Arthritis and rheumatism, 1995, 38, 44; A. Zanin-Zhorov, et al., J. Clin. Invest, 2006, 116, 2022). In co-culture experiments, CD4⁺CD25⁺ Teff cells were treated or not, washed, and added at different ratios (1:9, 1:3 or 1:1) to CD4⁺CD25⁻ Teff cells. The cells were co-cultured on anti-CD3 mAb pre-coated 24-well plates for 24-48 hr (cytokine secretion), or 96 hr (proliferation). Cytokine secretion was determined by ELISA as previously described A. Zanin-Zhorov, et al., ibid., 2006), using Human IFN-gamma Cytose™ (Biosource; Camarillo, Calif.). Proliferation was assessed by Alamar Blue™ assay (Invitrogen) as previously described (S. A. Ahmed, et al., J. immunological Methods, 1994, 170, 211-224).

SiRNA duplexes (siRNAs) were synthesized and purified by Qiagen Inc as described in the literature (K. K. Srivastava, et al., J. Biol. Chem. 2004, 279, 29911). The PKC-theta target sequences were: siRNA1 (5′-AAACCACCGTGGAGCTCTACT-3′) and siRNA2 (5′-AAGAGCCCGACCTTCTGTGAA-3′); control siRNA was purchased from Qiagen (1027281). Transfections of freshly purified T cells were performed using the human T cell Nucleofector kit (Amaxa Biosystems). Transfected cells were cultured in RPMI 1640 containing 10% FCS on immobilized anti-CD3 antibodies for 48-72 hours. Tranfection efficiency was controlled by evaluating PKC-theta levels using Western Blot analysis.

For T cell transfer model of colitis, we intravenously injected C57BL/10.PL TCR alpha^{-/-}beta^{-/-} mice with 5×10⁵ sorted CD4⁺CD25⁻CD45RB⁺ T cells alone or in combination with 0.125×10⁵ of CD4⁺CD25⁺ T cells that were pretreated or not as indicated. Disease progression was monitored by body weight loss, diarrhea and histology analysis as previously described (F. Powrie, et al., J. Exp. Med. 1996, 183, 2669). P values were determined by Mann-Whitney test or two-tailed t-test by using the GraphPad Prism software (San Diego, Calif.).

The PKC-theta inhibitors used and their IC₅₀'s are shown in Table 1 below. The preparation of these compounds and the luciferase assay used to determine the IC₅₀ for inhibition of the kinase activity of PKC-theta are described in US Patent Application Publication number 2005/0124640. For the in vitro and ex vivo assays above, compounds were dissolved in DMSO. T cells were pretreated with indicated concentrations of the inhibitors or DMSO control for 30 min at 37° C. and washed three times.

TABLE 1

| | | PKC theta Inhibition |
|---|---|---|
| Structure | Cpd Number | IC₅₀ (nM) |
| 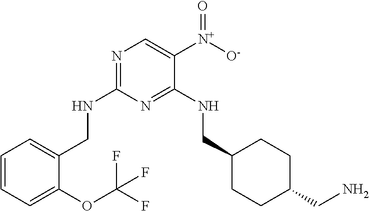 | Ia | 0.7 |
| 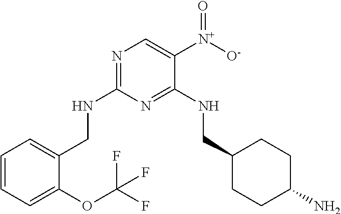 | Ib | 1.0 |
| 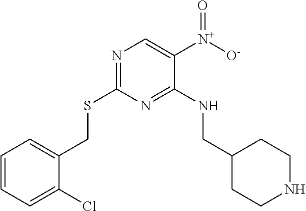 | Ic | 8 |
| 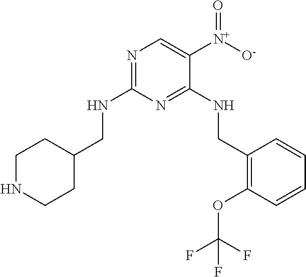 | Id | 300 |
| 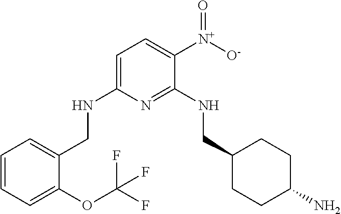 | Ie | 1000 |

1. A method of treating an immunological disorder or atherosclerosis in a patient comprising treating blood from the patient with an inhibitor of PKC-theta ex vivo and then re-administering the treated blood to the patient.

2. A method according to claim 1, wherein the patient has an immunological disorder.

3. A method according to claim 1, wherein the patient has atherosclerosis.

4. A method according to claim 1, wherein the leukocyte fraction from the patient blood is isolated and treated with an inhibitor of PKC-theta ex vivo and then re-administered to the patient.

5. A method according to claim 1, wherein the Treg cells from the patient blood are isolated and treated with an inhibitor of PKC-theta ex vivo and then re-administered to the patient.

6. A method according to claim 1, wherein the Treg cells from the patient blood are isolated, induced to grow to generate larger numbers of Treg cells and treated with an inhibitor of PKC-theta ex vivo and then re-administered to the patient.

7. A method according to claim 6, wherein the patient has an immunological disorder.

8. A method according to claim 6, wherein the patient has atherosclerosis.

9. A method according to claim 1, wherein peripheral blood mononucular cells and T-cells are separated by plasmapheresis from blood isolated from the patient having an immunological disorder and are treated with an inhibitor of PKC-theta ex vivo and then infused back into the patient.

10. A method according to claim 1, wherein peripheral blood mononucular cells and T-cells are separated by plasmapheresis from blood isolated from the patient having atherosclerosis and are treated with an inhibitor of PKC-theta ex vivo and then infused back into the patient.

11. A method according to claim 1, wherein the PKC-theta inhibitor is any inhibitor of PKC-theta disclosed in U.S. Pat. No. 7,550,473.

12. A method according to claim 1, wherein the PKC-theta inhibitor is a compound of formula (I) 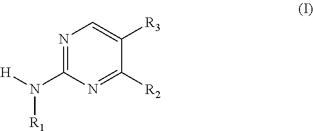
wherein:
R₁ is aryl-C₁₋₄ alkyl or heteroaryl-C₁₋₄alkyl, wherein in each of the C₁₋₄alkyl groups a methylene group may optionally be replaced by —NHC(O)— or —C(O)NH—, and wherein each of the C₁₋₄alkyl groups is optionally substituted by an oxo group or one or more C₁₋₃alkyl groups wherein two alkyl substituents on the same carbon atom of a C₁₋₄alkyl group may optionally be combined to form a C₂₋₅ alkylene bridge, and wherein the aryl group is optionally substituted on adjacent carbon atoms by a C₃₋₆alkylene bridge group wherein a methylene group is optionally replaced by an oxygen, sulfur or —N(R₆)—;
or R₁ has the following structure: 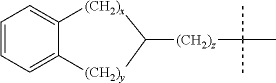
wherein x and y are independently 0, 1, 2 or 3, provided that x+y is 2 to 3, and z is 0 or 1;
wherein “heteroaryl” is defined as pyridyl, furyl, thienyl, pyrrolyl, imidazolyl, or indolyl;
wherein each R₁ group is optionally substituted by one or more of the following groups: C₁₋₆alkyl, Cl, Br, F, nitro, hydroxy, CF₃, —OCF₃, —OCF₂H, —SCF₃, C₁₋₄alkyloxy, C₁₋₄alkylthio, phenyl, benzyl, phenyloxy, phenylthio, aminosulfonyl, or amino optionally substituted by one or two C₁₋₃alkyl groups;
R₂ is selected from the following groups: 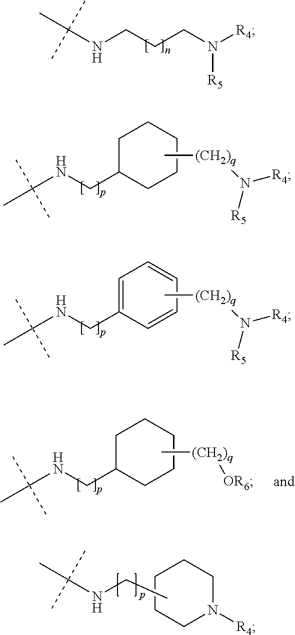
wherein:
n is an integer from 5 to 7;
p is an integer from 1 to 2;
q is an integer from 1 to 2;
R₄ and R₅ are each independently selected from hydrogen, C₁₋₆alkyl, arylC₁₋₆alkyl, or amidino;
R₆ is hydrogen;
R₃ is Br, Cl, F, cyano or nitro;
or a tautomer, pharmaceutically acceptable salt or solvate thereof.

13. A method according to claim 1, wherein the PKC-theta inhibition is achieved by siRNA or shRNA mediated suppression of PKC-theta and comprising treating blood from the patient with siRNA or shRNA ex vivo and then re-administering the treated blood to the patient.

14. A method according to claim 13, wherein the Treg cells from the patient blood are isolated and treated with siRNA or shRNA ex vivo and then re-administered to the patient.

15. A method according to claim 1, wherein the immunological disorder is selected from inflammatory diseases, autoimmune diseases, organ and bone marrow transplant rejection and other disorders associated with T cell mediated immune response, including acute or chronic inflammation, allergies, contact dermatitis, psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection) and systemic lupus erythematosus.
